(19) United States
(12) Patent Application Publication
(10) Pub. No.: US 2015/0166445 A1
(43) Pub. Date: Jun. 18, 2015

(54) DEHYDRATION OF DILUTIONS OF COMPOUNDS FORMING AN AZEOTROPE WITH WATER

(71) Applicant: AKZO NOBEL CHEMICALS INTERNATIONAL B.V., AMERSFOORT (NL)

(72) Inventor: Anton Alexandru Kiss, Arnhem (NL)

(21) Appl. No.: 14/404,283

(22) PCT Filed: Jun. 3, 2013
(86) PCT No.: PCT/EP2013/061335

§ 371 Date: Nov. 26, 2014

Related U.S. Application Data
(60) Provisional application No. 61/655,597, filed on Jun. 5, 2012.

(30) Foreign Application Priority Data
Jun. 5, 2012 (EP) 12170856.4

(51) Int. Cl. C07C 29/82; B01D 3/14; B01D 3/36

(52) U.S. Cl. CPC C07C 29/82; B01D 3/36; B01D 3/141

(57) ABSTRACT

A process and a column configuration for dehydration of an aqueous dilution of a compound forming an azeotrope with water, such as raw grade bioethanol, formic acid or chloroform, to form a concentrate with a concentration above azeotropic level. A preconcentration section (26, 40, 55) with a reboiler (29, 42, 57) and an extractive distillation section (22, 41, 52) are thermally coupled. The aqueous dilution is fed to the preconcentration section, where it is separated into water and a preconcentrate. The water is discharged via the reboiler, and the preconcentrate is fed to the extractive distillation section. A solvent is fed to the extractive distillation section at a higher level than the preconcentrate. In the extractive distillation section the final concentrate is separated from a mixture of the solvent and water.

The present invention relates to a process and a column configuration for dehydration of an aqueous dilution of a compound forming an azeotrope with water, to form a concentrate with a concentration above azeotropic level. In a preferred embodiment, the invention relates to the dehydration of aqueous mixtures of ethanol, such as bioethanol, to provide concentrates of a desired purity, for instance to be used as a fuel or fuel additive. In another preferred embodiment, the invention relates to the dehydration of aqueous mixtures of formic acid or chloroform.

Bioethanol is typically produced by subsequent saccharification and fermentation of biomass, such as lignocellulosic biomass or biomass from sugar canes and/or corn. The fermentation generally results in an aqueous mixture of 5-12 wt % bioethanol. For use as a fuel or fuel additive, bioethanol must have a purity of 99.6-99.8 wt % (see US Standard ASTM D 4806 and European standard EN 15376).

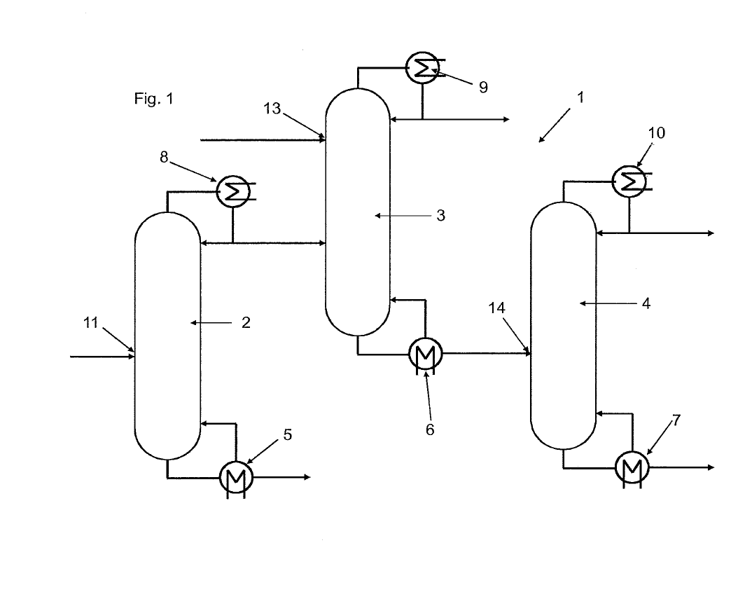

The binary azeotrope of an ethanol-water mixture has an ethanol content of 95.63 wt % ethanol. Consequently, maximum purity obtainable by regular distillation is 95.63 wt %. To obtain a bioethanol fraction with the standard required ethanol concentration of 99.8 wt %, the dehydration process is presently carried out in a sequence of steps, including a first pre-concentration step in a distillation column, typically resulting in a purity of about 92-94 wt %. In a second step the ethanol is dehydrated to the desired degree of ethanol concentration, for instance by pervaporation, adsorption, pressure swing distillation, extractive distillation or azeotrope distillation or combinations thereof. If extractive or azeotropic distillation is used, the used solvent must be recovered and dehydrated. FIG. 1 of the accompanying drawings schematically shows such a three-step bioethanol dehydration according to present-day state of the art.

Such multi-step processes require high energy consumptions. In the article by A. Kiss and D. J. P. C Suszwalak, “Enhanced bioethanol dehydration by extractive and azeotropic distillation in dividing-wall columns”, Separation and Purification Technology, 86, p. 70-78, (2012), it has been proposed to combine the second step (extractive distillation of a preconcentrated ethanol fraction) with the third step (solvent recovery) in a dividing top wall column. The top section and middle sections of this column are divided by a vertically extending dividing wall separating the feed side from the discharge side. The bottom section is undivided and is provided with a single reboiler. At the inlet side of a middle section a preconcentrated bioethanol is fed to the column. At a higher level ethylene glycol is fed to the column. Ethanol rises to the top section of the inlet side, where it is discharged via a first condenser. A mixture of water and ethylene glycol flows down to the bottom section, where water is vaporized for separation from the ethylene glycol which is discharged via the reboiler at the bottom section, while the water is discharged via a condenser at the top section of the discharge side of the column. However, to obtain the desired ethanol concentration this system still requires a separate preconcentration step, which is in fact the most energy-intensive part of the process.

It is an object of the invention to design a dehydration process for aqueous dilutions of a compound forming an azeotrope with water, such as raw grade bioethanol, formic acid, and chloroform, resulting in dehydrated fractions of the required level of concentration, requiring less consumption of energy.

The object of the invention is achieved with a process using a preconcentration section with a reboiler and an extractive distillation section, the preconcentration section being thermally coupled to the extractive distillation section. The aqueous diluted stream is fed to the preconcentration section, where it is separated into water and a preconcentrate. Separated water is discharged via the reboiler. The preconcentrate is fed to the extractive distillation section. A solvent is fed to the extractive distillation section at a higher level than the preconcentrate. In the extractive distillation section the final concentrate is separated from a mixture of the solvent and water.

Substantial energy savings can be achieved by thermally coupling the preconcentration section and the extractive distillation section. The term “thermally coupled” means that there is two-way communication between the columns (see e.g. Agrawal R., “More operable arrangements of thermally coupled distillation columns”, AlChE, USA, 1999; Fidkowski Z., Krôlikowski L., “Minimum energy requirements of thermally coupled distillation systems”, AlChE Journal, 33 (1987), 643-653). More particularly, there is also a back coupling of, in this case, the extractive distillation section to the preconcentration section. Thermally coupled column configurations comprise interconnecting streams (at least one in the vapour phase and one in the liquid phase) between columns or between separated sections of a column. Each interconnecting stream replaces a condenser or a reboiler from one of the columns or column sections.

In a specific embodiment, the mixture of solvent and water leaving the extractive distillation section is transferred to a solvent recovery section, where solvent is separated from the water by distillation and discharged via a second reboiler. Optionally, the separated solvent can be returned to the extractive distillation section for re-use.

Although this configuration makes use of two reboilers, it was surprisingly found that this results in substantial overall energy savings compared to prior art systems. According to rigorous simulation calculations, the energy savings typically lie between 10% and 20% and for some cases even above 20%. Similar savings of about 20% are possible also with the capital investment cost, while the overall plant CO₂ footprint can be substantially reduced due to the reduced number of required equipment units.

In a specific embodiment, the column configuration comprises a single column with a dividing wall dividing a middle section of the columns between a feed side, forming the preconcentration section, and a discharge side, wherein an undivided top section of the column forms an extractive distillation section.

Optionally, the dividing wall column comprises an undivided bottom section forming a solvent recovery section with a second reboiler. To obtain sufficiently pure water, the preconcentration section can comprise a water draw-off line to the first reboiler at a location above the level of the lower edge of the dividing wall. Alternatively, the dividing wall may also divide the bottom section, wherein the solvent recovery section is formed by a separate column downstream of the dividing wall column. The separate solvent recovery column can for instance be connected to the discharge side of the bottom section of the dividing wall column via a reboiler.

The diluted fraction of the compound can for example be fed to the preconcentration section at the level of a top edge of the dividing wall. The solvent can for instance be fed to the column at a higher level than the feed of the aqueous dilution.

In an exemplary embodiment, the column can comprise at least 30 theoretical stages, wherein the undivided top section comprises at least at least 30% of the theoretical stages, while the undivided bottom section comprises at least 10% of the theoretical stages.

In an alternative embodiment, the preconcentration section and the extractive distillation section can be separate columns thermally coupled by an upper vapour line transporting preconcentrated compound to an upper section of the extractive distillation section, and a vapour return line returning water vapour from the bottom section of the extractive distillation section to the preconcentration section.

In such a configuration the extractive distillation may for example comprise at least thirty theoretical stages, wherein the upper vapour line extends from a top stage of the preconcentration section to the level of any one of the 25^{th}-30^{th} stages of the extractive distillation section. In such a configuration the solvent can for example be fed to the extractive distillation section at a level above the upper vapour line. The vapour return line can for instance extend from the level of one of the ten lowest theoretical stages of the extractive distillation section to the bottom section of the preconcentration section.

The aqueous dilution of a compound which is dehydrated using the process for dehydration according to the present invention is preferably selected from the group consisting of an aqueous ethanol fraction, an aqueous propanol fraction, an aqueous butanol fraction, an aqueous allyl alcohol fraction, an aqueous formic acid fraction, an aqueous propionic acid fraction, an aqueous butyric acid fraction, an aqueous nitric acid fraction, an aqueous hydrofluoric acid fraction, an aqueous chloroform fraction, an aqueous methylene chloride fraction, an aqueous ethylene chloride fraction, an aqueous propylene fraction, an aqueous 1,2-dichloroethane fraction, an aqueous methyl acetate fraction, an aqueous propyl acetate fraction, an aqueous ethyl nitrate fraction, an aqueous acetone fraction, an aqueous methyl ethyl ketone fraction, an aqueous benzene fraction, an aqueous cyclohexane fraction, an aqueous diethyl ether fraction, an aqueous tetrahydrofuran fraction, an aqueous acetonitrile fraction, an aqueous chloral fraction, an aqueous methyl tert-butyl ether fraction, an aqueous triethyl amine fraction, an aqueous di-isopropyl amine fraction, an aqueous dimethyl acetal fraction, an aqueous 1,3-dioxolane fraction, an aqueous propionaldehyde fraction, an aqueous isoveralaldehyde fraction, an aqueous acroleine fraction, an aqueous 2-methyl 2-propanol, and an aqueous n-methylbutyl amine fraction.

More preferably, the aqueous dilution of a compound which is dehydrated using the process for dehydration according to the present invention is selected from the group consisting of an aqueous ethanol fraction, an aqueous propanol fraction, an aqueous butanol fraction, an aqueous allyl alcohol fraction, an aqueous formic acid fraction, an aqueous propionic acid fraction, an aqueous butyric acid fraction, an aqueous hydrofluoric acid fraction, an aqueous chloroform fraction, an aqueous methylene chloride fraction, and an aqueous ethylene chloride fraction.

Even more preferably, the aqueous dilution of a compound which is dehydrated using the process for dehydration according to the present invention is selected from the group consisting of an aqueous ethanol fraction, an aqueous formic acid fraction, and an aqueous chloroform fraction.

The disclosed process is particularly useful to dehydrate aqueous ethanol fractions, such as raw grade bioethanol. Such a dehydration process can be carried out at atmospheric pressure, or optionally at higher or lower pressures, if so desired.

For the sake of clarity it is noted that “an aqueous fraction” of a compound means an aqueous dilution of a compound.

The temperature in the dividing wall column can for instance range from about 60-120° C. at the top to about 160-240° C. at the bottom section with a sharp increase from about 80-140° C. at the level of the lower edge of the dividing wall to about 160-240° C. at the lowest point of the column (depending on the boiling point of the solvent used). The temperature at the preconcentration section can for instance range from about 60-120° C. at the level of the top edge of the dividing wall to about 80-140° C. at the level of the lower edge of the dividing wall. Any other temperature profiles can also be used, to be determined by routine optimization on the basis of the composition of the feed and the required concentration of the dehydrated compound and the operating pressure used.

Extractive distillation takes place in the extractive distillation section by adding the solvent to the preconcentrate. As the solvent (also sometimes denoted an extractive agent), any liquid can be used which has a boiling point which is higher than the boiling point of water and of the compound to be dehydrated (at the same pressure), relatively non-volatile component (very low or negligible vapour pressure, defined here as lower than 10 mmHg at 20° C.) that is completely miscible with the preconcentrate at distillation conditions and that does not form an azeotrope with the components of the preconcentrate. For example, suitable solvents for the extractive distillation of ethanol include ethylene glycol, propylene glycol, and glycerol. As described above, other solvents with a higher boiling point than water and ethanol itself and not forming an azeotrope with water or ethanol, can also be used, provided they are miscible with the preconcentrate under distillation conditions. Examples of other suitable solvents for the dehydration according to the invention of aqueous ethanol fractions include certain hyperbranched polymers and certain ionic liquids. Suitable solvents (extractive agents) for the extractive distillation of, for instance, formic acid or chloroform include isopropanol, t-butanol, isobutanol, n-propyl acetate, n-butyl acetate, 1,2-butanediol, diisobutyl ether, 3-nitrotoluene, 4-methyl-2-pentanone, propoxypropanol or (although less preferred) a combination of these components.

The object of the invention is also achieved with a column configuration for the dehydration of an aqueous dilution of a compound forming an azeotrope with water, to a concentration above azeotropic level, the column configuration comprising at least three sections including:
a preconcentration section with a first reboiler,
an extractive distillation section with a condenser,
a solvent recovery section with a second reboiler,

wherein the column configuration comprises a column encasing at least two of the three sections. The preconcentration section is thermally coupled to the extractive distillation section by a top vapour passage, while the extractive distillation section is provided with at least one feed of a solvent at a level above the top vapour passage and with a condenser at its top section.

The column can for example be a dividing wall column with a dividing wall dividing at least a middle section of the column between a feed side, forming the preconcentration section, and a discharge side, wherein a top section is undivided. In that case, the bottom section may be undivided forming a solvent recovery section or it may be divided between an inlet side, forming part of the preconcentration section, and a discharge side connected, e.g., via a reboiler, to a separate next column forming the solvent recovery section.

The column or columns will typically comprise a plurality of theoretical stages. In the specific embodiment of the dividing wall column, the column may for instance have 10-50 theoretical stages, e.g. 30-45 stages filled with (structured) packing internals and/or trays. Such packing can comprise solid or hollow bodies of predetermined size, shape, and configuration used as column internals to provide surface for liquid to allow mass transfer at the liquid-vapour interface during countercurrent flow of two phases. With a structured packing, individual members have a specific orientation relative to each other and to the column axis. Structured packing material is usually made of thin metal foil, expanded metal, plain sheet metal, and/or woven wire screen stacked in layers or as spiral bindings, but other packing types can also be used. Trays can be used instead of packing or in addition thereto. Such a tray typically comprises a decking or contacting deck with means to deliver liquid to the tray from a next higher tray and to remove liquid for passage to the next lower tray. The liquid removed from the tray flows down through a down-comer of the tray. Vapour generated in a lower portion of the column passes upward through perforations in the decking, while liquid flows downward from tray to tray countercurrently to the vapour.

Particularly suitable are the following types of packing and/or trays: Sulzer Mellapak®(Plus), CY, BX(Plus), I/C/P/R-ring, Pall rings, Cascade MiniRing®, Raschig® rings, Raschig® Super-Ring/Pak, Intalox® (Ultra), Berl® saddles, Nutter® rings, hollow fibers, VGPlus trays, SuperFrac trays, (wire-mesh-packed) sieve trays, bubble cap trays, or valve trays.

The invention will be further explained under reference to the accompanying drawings.

FIG. 1: shows schematically a prior art column configuration for the dehydration of bioethanol;

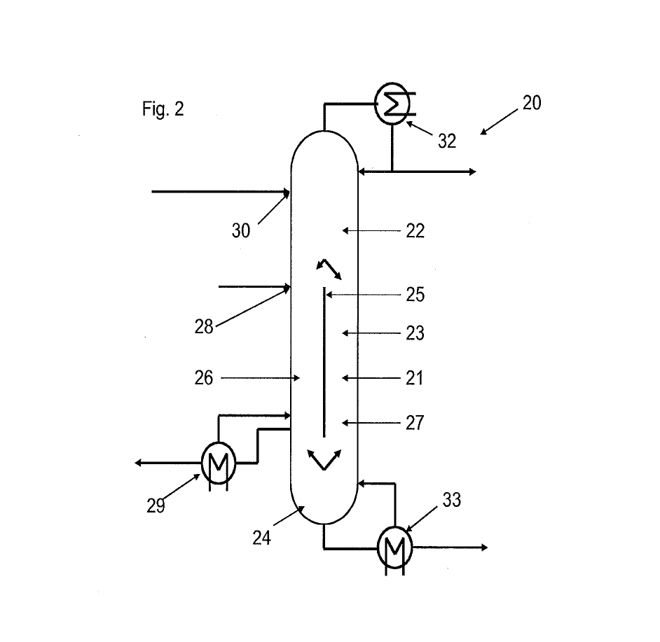

FIG. 2: shows a first exemplary embodiment of a column configuration according to the present invention;

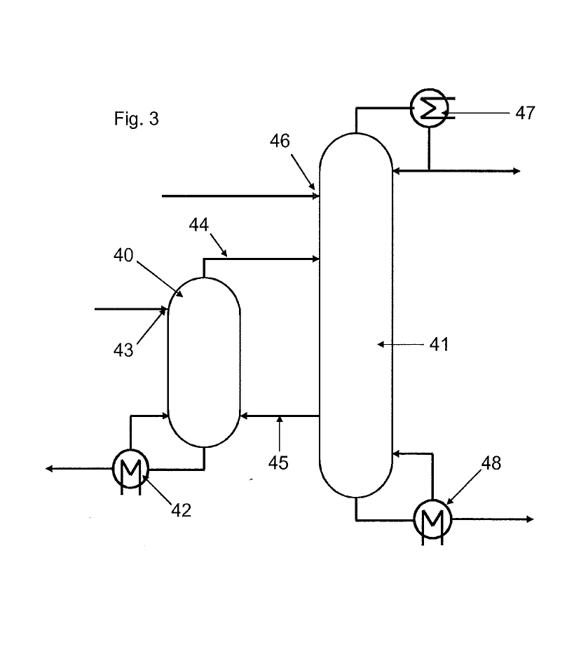

FIG. 3: shows a second exemplary embodiment of a column configuration according to the present invention;

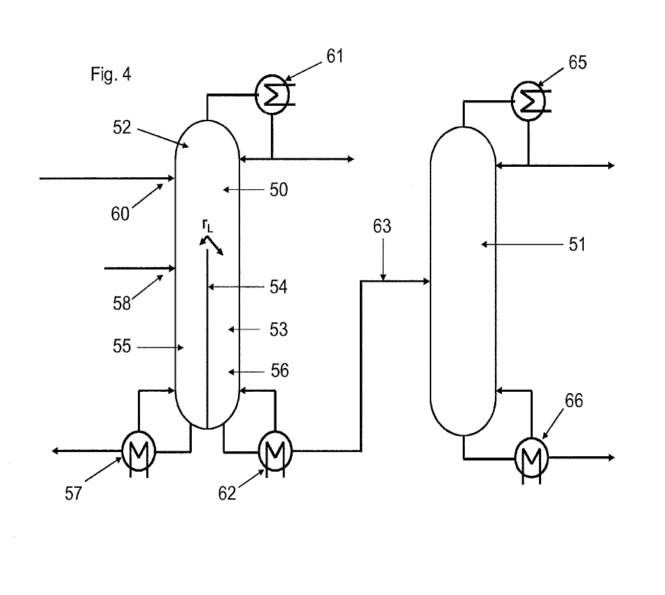

FIG. 4: shows a third exemplary embodiment of a column configuration according to the present invention;

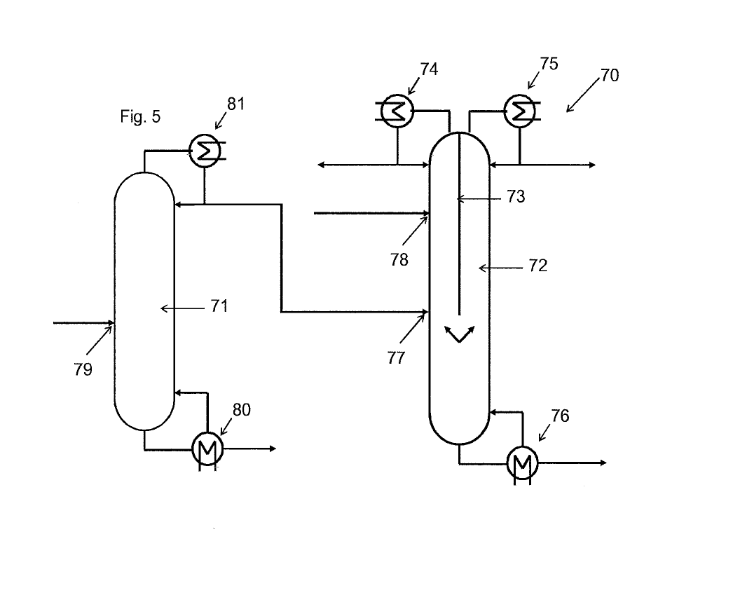

FIG. 5: shows the combination of a conventional preconcentration distillation column (such as the first one shown in configuration of FIG. 1) and a prior art extractive dividing top-wall column.

FIG. 1 shows a prior art column configuration 1 used in the Comparative Example hereafter. The column configuration 1 comprises a series of three distillation columns 2, 3, 4 all being provided with a reboiler 5, 6, 7 at their respective bottom sections, and a condenser 8, 9, 10 at their respective top sections. The first column 2 is a preconcentration column. A feed comprising an aqueous dilution of a compound forming an azeotrope with water, such as ethanol, is fed to the column 2 via an inlet 11. Water is discharged via the reboiler 5, while a concentrate of the compound is discharged via the condenser 8 and fed to the lower half of the second column 3 via an inlet 12. A high boiling solvent is fed to the second column 3 via an inlet 13 at a level above the feed inlet 12 for extractive distillation of the concentrated compound.

A purified fraction of the compound is discharged via the condenser 9 of the second column. A mixture of water and solvent is discharged via the reboiler 6 and fed to the third column via an inlet 14. In the third column 4 water and solvent are separated by distillation. Water is discharged via the condenser 10, while recovered solvent is discharged via the reboiler 7. The recovered solvent can for example be reused in the second column 3.

FIG. 2 shows a first exemplary embodiment of a column configuration 20 according to the present invention. The configuration includes a single dividing wall column 21 with a top section 22, a middle section 23, and a bottom section 24. The middle section 23 is divided by a vertical dividing wall 25 into a preconcentration section 26 and a solvent recovery section 27. A feed inlet 28 opens into the preconcentration section 26 at the level of the top edge of the dividing wall 25. A first reboiler 29 is connected to the preconcentration section 26 at the level of the lower edge of the dividing wall 25.

A solvent inlet 30 opens into the top section 22 at a position above the preconcentration section 26. Extractive distillation takes place in the top section 22. A purified fraction of the compound is discharged via a condenser 32 at the top of the column 20. Part of the condensate is recycled as a reflux to the column 20, while the rest is collected as a distillate product. A mixture of solvent and water flows down via the section 23 using a liquid split ratio of 0:1 to the bottom section, where it is distilled. Water vapour goes up to the first reboiler 29, where it condenses due to the countercurrent aqueous dilution fed to the column 20 via the feed inlet 28. The condensed water is subsequently discharged to the first reboiler 29. Part of the water is collected as a liquid product, while the rest is vaporized and recycled to the column 20. Recovered solvent is discharged via a second reboiler 33 at the bottom of the dividing wall column 20 and optionally recycled in the process via the solvent inlet 30.

A second exemplary embodiment is shown in FIG. 3. This embodiment comprises two thermally coupled columns 40, 41. The first column 40 forms a preconcentration section with a first reboiler 42 and a feed inlet 43 for the supply of an aqueous dilution of a compound to be purified. An upper vapour line 44 connects the top of the first column 40 to the upper half of the second column 41. A vapour return line 45 connects the lower half of the second column 41 with a bottom section of the first column 40. In the first column the aqueous dilution of the compound to be purified is preconcentrated. Separated water is discharged via the reboiler 42. An aqueous concentrate of the compound flows as a vapour via vapour line 44 to the second column 41. A solvent feed 46 opens into the second column at a level above the upper vapour line 44. A high boiling solvent is fed to the second column 41 via the solvent inlet 46. The second column 41 comprises a condenser 47 at its top and a reboiler 48 at its bottom. The compound is separated from the water by extractive distillation and discharged via the condenser 47. A mixture of water and solvent goes down to the bottom section 39. Here, liquid solvent is discharged via the reboiler 48 while vapour phase water is returned to the first column 40 via the vapour return line 45.

A further possible embodiment is represented schematically by FIG. 4 showing a divided first column 50 and an undivided second column 51. The first column 50 comprises an undivided top section 52. The column 50 further comprises a middle and bottom section 53 divided by a vertically extending dividing wall 54 into a feed side 55 and a discharge side 56. In this embodiment the dividing wall 54 extends to the bottom of the column 50, thereby physically dividing the bottom section, although one or more openings can be provided if so desired. The feed side 55 functions as a preconcentration section with a first reboiler at its bottom 57. A feed inlet 58 is connected to the feed side 55 at or near the level of the top edge of the dividing wall 54. A solvent feed 60 opens into the undivided top section 52 at a distance above the bioethanol inlet 58. A condenser 61 is arranged at the top section of the first column 50. The discharge side 56 of the bottom section 53 is provided with a reboiler 62. Since the dividing wall 54 extends to the bottom of the column 50, the liquid streams flowing through the first and second reboilers 57, 62 cannot remix in the split bottom section of the column 50.

The reboiler 62 is connected to the second column 51 via a line 63 opening into the second column 51 at about half the height of the second column 51. The second column 51 is a distillation column with a condenser 65 at its top and a reboiler 66 at its bottom.

In use an aqueous dilution of a compound is fed to the first column 50 via the inlet 58. Water flows down and is discharged via the first reboiler 57. Preconcentrated compound vaporizes upwardly against a counterflow of a solvent fed to the column 50 via solvent inlet 60. The solvent extracts water from the preconcentrate and flows down. A mixture of solvent and water is discharged via the second reboiler 62, while purified ethanol is collected via the condenser 61 at the top of the column 50. The mixture of solvent and water is fed to the second column 51, where water is separated by distillation and discharged via the condenser 65 of the second column 51. Separated solvent is collected via the third reboiler 66. Optionally, the recovered solvent is returned to the first column 50 via the solvent inlet 60.

The following Example and Comparative Examples 1 and 2 were generated using Aspen Plus® simulation software using the RADFRAC unit with RateSep (rate based) model. NRTL property method was used due to the presence of a non-ideal mixture containing polar elements. The column configurations in the Example and Comparative Examples 1 and 2 were both optimized in terms of minimal energy demand using the sequential quadratic programming (SQP) module of Aspen Plus®. In the Example and Comparative Examples 1 and 2, a raw grade bioethanol is dehydrated and purified using ethylene glycol as a solvent for extractive distillation.

### COMPARATIVE EXAMPLE 1

An aqueous 10 wt % dilution of bioethanol was fed to the column configuration of FIG. 1 with a production rate of 100 kt/y (equivalent to 12,500 kg/hr of raw grade bioethanol feed, assuming an 8,000 hr/y operation). In the first column 2 used for the preconcentration step, water is discharged from the bottom section via the reboiler with a purity of about 99.99 wt %, while the bioethanol concentration of the mixture was increased by distillation to a near-azeotropic composition with an ethanol content of about 93.5 wt %. This preconcentrate stream from the first column 2 is fed to the second column 3. Ethylene glycol (20,793 kg/hr) is fed to the second column 3 as a solvent (or mass separation agent) for extractive distillation of the ethanol preconcentrate. Ethanol with a purity of 99.8 wt % is discharged via the condenser 9, while a mixture of ethylene glycol and water is discharged via the reboiler 6 and subsequently fed to the third column 4, where water is separated from the ethylene glycol by distillation, e.g., recovering over 99.99 wt % of the solvent.

In the calculations, the first column 2 has 30 theoretical stages, the feed line 11 being at the level of the 21^{st} stage (counting top-down). The second column 3 has 17 stages, with the solvent feed 13 being at the level of the 4^{th} stage and the concentrate feed line being at the level of the 11^{th} stage. The third column 4 has 16 theoretical stages, with the feed line 14 for the supply of the ethylene glycol-water mixture being at the level of the 8^{th} stage. All columns 2, 3, 4 are operated at atmospheric pressure at the condenser level in a normal distillation window outside flooding region.

The temperature in the preconcentration column ranges from 78° C. at the level of the top to about 100° C. at the bottom. The temperature in the second column ranges from 80° C. at the top to about 160° C. at the bottom. In the third column the temperature ranges from about 100° C. at the top to about 200° C. at the bottom. The reflux ratio R:D, conventionally defined as the molar ratio of the liquid reflux R returned to the column, and the liquid distillate product D, both per unit of time, is 2.9 in the first column, 0.17 in the second column, and 0.6 in the third column. The heat requirement for the three columns is 23,882 kW, 5,574 kW, and 1,454 kW, respectively (making 30,910 kW in total), which illustrates that the preconcentration step consumes the largest part of the required energy.

It was calculated that the specific energy requirement of this column configuration is 2,470 kW·h per ton bioethanol. CO₂-emission was calculated to be 345.77 kg CO₂/(h·ton bioethanol).

### COMPARATIVE EXAMPLE 2

This second Comparative Example considers the combination of a conventional preconcentration distillation column (such as the first one shown in configuration of FIG. 1) and an extractive dividing top-wall column (E-DWC) as described by A. Kiss and D. J. P. C Suszwalak, “Enhanced bioethanol dehydration by extractive and azeotropic distillation in dividing-wall columns”, Separation and Purification Technology, 86, p. 70-78, (2012), used for combined dehydration and solvent recovery.

FIG. 5 shows such a set-up of a preconcentration distillation column 71 and a E-DWC 72 (configuration 70). The dividing top wall 73 extends from the top of the column 72 dividing the top and middle sections into a feed side and a discharge side, both having a condenser 74, 75 at the respective tops. The bottom section of the column is undivided and is provided with a reboiler 76. Preconcentrate from the first column is fed to the feed side via the preconcentrate inlet 77 of the split section of the dividing top wall column 72. Ethylene glycol is fed via an inlet 78 above the preconcentrate inlet 77. Purified ethanol is discharged via the first condenser 74 at the feed side of the split sections, while water is discharged via the second condenser 75 at the discharge side of the split sections. Recovered ethylene glycol is discharged via the reboiler 76 at the undivided bottom of the column.

In this Comparative Example, the preconcentration column 71 and the dividing top wall column 72 are both operated at atmospheric pressure at the condenser level, in a normal distillation window outside flooding region. An aqueous 10 wt % dilution of bioethanol was fed via inlet 79 to the first column 71 used for the preconcentration step, water is discharged from the bottom section via the reboiler 80 with a purity of 99.99 wt %, while the bioethanol concentration of the mixture was increased by distillation to a near-azeotropic composition with an ethanol content of 93.5 wt %. This preconcentrate stream from the first column 71 is fed to the second column 72 via the condenser 81 and the preconcentrate inlet 77. Ethylene glycol (amounting to 1.9 solvent to feed molar ratio) is fed to the second column as a solvent for extractive distillation of the ethanol preconcentrate. Ethanol with a purity of 99.8 wt % is discharged via one condenser (feed side), water % is discharged via the second condenser (discharge side), and ethylene glycol is discharged via the reboiler, e.g., recovering over 99.98 wt % of the solvent.

For the sake of clarity, configuration 70 differs from the column configuration according to the present invention in that columns 71 and 72 are not thermally coupled. One water outlet is removed as bottom product of column 71, and the other water outlet is removed as top distillate product, discharged via the condenser 74, whereas in the column configuration according to the present invention, water is discharged only as side product via a reboiler.

In the calculations, the first column 71 has 30 theoretical stages, the feed line being at the level of the 21^{st} stage (counting top-down). The extractive dividing top wall column has 20 stages, with the solvent feed being at the level of the 3^{rd} stage and the preconcentrate feed line being at the level of the 13^{th} stage. The dividing wall 73 partitioning the top section extends from the top of the column downwardly until stage 16.

The temperature in the preconcentration column ranges from 78° C. at the level of the top to about 100° C. at the bottom. The temperature in the extractive dividing top wall column ranges from 78° C. and 100° C. at the top of the left and right sections, to about 200° C. at the bottom. The reflux ratio is 2.9 in the first column and 0.27 and 0.2 on the feed and discharge sides, respectively, in the dividing top wall column. It was calculated that the specific energy requirement of this column configuration is 1,910 kW·h/ton (for the preconcentration column) and 460 kW·h/ton (for the dividing top wall column), thus leading to a total of 2,370 kW·h per ton bioethanol for this process.

### EXAMPLE

The same bioethanol feed (12,500 kg/hr) is fed to the dividing wall column of FIG. 2 with the same production rate (100 kt/y). The column is operated at atmospheric pressure at the condenser level. Ethylene glycol was used as the solvent with a flow rate of 20,793 kg/hr. Ethanol of 99.8 wt % was discharged via the condenser. Water (99.9 wt %) was discharged via the first reboiler at the feed side of the column, while ethylene glycol (99.99 wt %) was recovered via the second reboiler at the bottom of the column.

In the calculations the dividing wall column has 42 theoretical stages, the highest 17 stages forming the top section, the lowest 8 stages forming the bottom section. The dividing wall extends from the 17^{th} stage to the 35^{th} stage. The aqueous raw grade bioethanol dilution is fed at the 18^{th} stage (feed side of the dividing wall), while the solvent feed line is at the level of the 4^{th} stage. The liquid split ratio above the partition wall is 0:1, while the vapour split ratio below the partition wall is 0.4:0.6 (feed vs. side section).

The temperature ranges from about 80° C. at the top to about 200° C. at the bottom section, with a sharp increase from 120° C. at the level of the lower edge of the dividing wall to about 200° C. at the lowest point. The temperature at the preconcentration section ranges from about 80° C. at the level of the top edge of the dividing wall to about 100° C. at the level of the lower edge of the dividing wall.

It was calculated that the total heat duty required is 25,775 kW, meaning that the specific energy requirement of this column configuration is 2,070 kW·h per ton bioethanol. CO₂-emission was calculated to be 288.31 kg CO₂/(h·ton bioethanol).

Accordingly, the specific energy requirement as well as the CO₂-emission of the configuration used in the first Comparative Example is a factor of about 1.2 times higher than the calculated specific energy requirement and the CO₂-emission of the configuration as used in the Example. Surprisingly, the specific energy requirement of the configuration used in the second Comparative Example is more than 1.14 times higher than the calculated specific energy requirement of the Example. Moreover, the investment costs are estimated to be about 20% lower than for the equipment used in the Example.

1. A process for dehydration comprising dehydrating an aqueous dilution of a compound forming an azeotrope with water to form a concentrate with a concentration above azeotropic level, using a preconcentration section with a reboiler and an extractive distillation section, the preconcentration section being thermally coupled to the extractive distillation section, wherein the aqueous dilution is fed to the preconcentration section, where it is separated into water and a preconcentrate, the water being discharged via the reboiler, and the preconcentrate being fed to the extractive distillation section, wherein a solvent is fed to the extractive distillation section at a higher level than the preconcentrate, wherein in the extractive distillation section the final concentrate is separated from a mixture of the solvent and water.

2. The process according to claim 1 wherein the mixture of solvent and water is transferred to a solvent recovery section where solvent is separated from the water by distillation and discharged via a second reboiler.

3. The process according to claim 2 wherein separated solvent is recycled to the extractive distillation section.

4. The process according to claim 1, wherein a single column is used with a dividing wall dividing a middle section of the column between a feed side, forming the preconcentration section, and a discharge side, wherein an undivided top section of the column forms the extractive distillation section.

5. The process according to claim 4 wherein the column comprises an undivided bottom section forming a solvent recovery section with a second reboiler.

6. The process according to claim 4 wherein the aqueous dilution of the compound is fed to the preconcentration section at the level of a top edge of the dividing wall.

7. The process according to claim 6 wherein the solvent is fed to the column at a higher level than the feed of the aqueous dilution.

8. The process according to claim 4 wherein the column comprises at least 30 theoretical stages, wherein the undivided top section comprises at least 30% of the theoretical stages, while the undivided bottom section comprises at least 10% of the theoretical stages.

9. The process according to claim 1, wherein the preconcentration section and the extractive distillation section are separate columns thermally coupled by an upper vapour line transporting preconcentrated compound to an upper section of the extractive distillation section, and a vapour return line returning water vapour from the bottom section of the extractive distillation section to the preconcentration section.

10. The process according to claim 9 wherein the extractive distillation section comprises at least 30 theoretical stages, and wherein the upper vapour line extends from a top stage of the preconcentration section to the level of any one of the 25^{th}-30^{th} stages of the extractive distillation section.

11. The process according to claim 10 wherein the solvent is fed to the extractive distillation section at a level above the upper vapour line.

12. The process according to claim 9, wherein the vapour return line extends from the level of one of the ten lowest theoretical stages of the extractive distillation section to the bottom section of the preconcentration section.

13. The process according to claim 1, wherein the aqueous dilution compound is selected from the group consisting of an aqueous ethanol fraction, an aqueous propanol fraction, an aqueous butanol fraction, an aqueous allyl alcohol fraction, an aqueous formic acid fraction, an aqueous propionic acid fraction, an aqueous butyric acid fraction, an aqueous nitric acid fraction, an aqueous hydrofluoric acid fraction, an aqueous chloroform fraction, an aqueous methylene chloride fraction, an aqueous ethylene chloride fraction, an aqueous propylene fraction, an aqueous 1,2-dichloroethane fraction, an aqueous methyl acetate fraction, an aqueous propyl acetate fraction, an aqueous ethyl nitrate fraction, an aqueous acetone fraction, an aqueous methyl ethyl ketone fraction, an aqueous benzene fraction, an aqueous cyclohexane fraction, an aqueous diethyl ether fraction, an aqueous tetrahydrofuran fraction, an aqueous acetonitrile fraction, an aqueous chloral fraction, an aqueous methyl tert-butyl ether fraction, an aqueous triethyl amine fraction, an aqueous di-isopropyl amine fraction, an aqueous dimethyl acetal fraction, an aqueous 1,3-dioxolane fraction, an aqueous propionaldehyde fraction, an aqueous isoveralaldehyde fraction, an aqueous acroleine fraction, an aqueous 2-methyl 2-propanol, and an aqueous n-methylbutyl amine fraction.

14. The process according to claim 13 wherein the solvent comprises ethylene glycol.

15. A column configuration for the dehydration of an aqueous dilution of a compound forming an azeotrope with water, to a concentration above azeotropic level, the column configuration comprising three sections including:
a preconcentration section with a first reboiler,
an extractive distillation section with a condenser,
a solvent recovery section with a second reboiler,
wherein the column configuration further comprises a column encasing at least two of the three sections, wherein the preconcentration section is thermally coupled to the extractive distillation section by a top vapour passage, the extractive distillation section being provided with at least one feed of a solvent at a level above the top vapour passage and with a condenser at its top section.

16. The column configuration according to claim 15 wherein the column is a dividing wall column with a dividing wall dividing at least a middle section of the column between a feed side, forming the preconcentration section, and a discharge side, wherein a top section is undivided.

17. The column configuration according to claim 16 wherein the column comprises an undivided bottom section forming a solvent recovery section and comprising a reboiler.

18. The column configuration according to claim 16 further comprising a separate column forming the solvent recovery section and wherein the dividing wall extends from the bottom of the dividing wall column, wherein the discharge side of the bottom comprises a reboiler and a line for transporting the mixture of solvent and water to the solvent recovery column.

19. The column configuration according to claim 15 wherein the preconcentration section and the extractive distillation section are separate thermally coupled columns.

20. The process according to claim 7 wherein the column comprises at least 30 theoretical stages, wherein the undivided top section comprises at least 30% of the theoretical stages, while the undivided bottom section comprises at least 10% of the theoretical stages.
